(19) United States
(12) Patent Application Publication
(10) Pub. No.: US 2018/0014960 A1
(43) Pub. Date: Jan. 18, 2018

(54) SHOULDER BRACE

(71) Applicants: Thomas SAWA, Mississauga (CA); Richard Philip SAWA, Columbus, OH (US)

(72) Inventors: Thomas SAWA, Mississauga (CA); Richard Philip SAWA, Columbus, OH (US)

(21) Appl. No.: 15/210,251

(22) Filed: Jul. 14, 2016

(51) Int. Cl. A61F 5/01

(52) U.S. Cl. CPC A61F 5/0118

(57) ABSTRACT

A shoulder brace uses an adjustable outer cuff for engaging an arm of a user and providing a lifting force. This force is redistributed to a body portion of the brace in a manner to reduce arm and shoulder strain that can be associated with arm or shoulder repetitive movement. The brace is particularly helpful for long periods of exposure to playing of musical instruments such as a guitar but is useful for other applications.

### FIELD OF THE INVENTION

The present invention relates to a shoulder brace and in particular relates to a shoulder brace designed to assist in distributing an arm load that is likely to be incurred for an extended period of time.

### BACKGROUND OF THE INVENTION

Shoulder braces are most commonly used to provide additional support or limit certain movements when a user has an existing condition such as torn or strained muscles. Often the braces are designed to limit, movement in a given direction to avoid further damage. It is desirable in this type of limiting movement shoulder brace to allow as much normal movement as possible while providing a restricted range of motion in one or more directions to avoid further damage or to avoid a specific movement.

In contrast to the limiting type shoulder braces, the present shoulder brace is designed to redistribute a particular force that will be encountered for an extended period due to a particular activity. For example playing of a guitar requires the user's lower hand to be placed over the guitar with the hand positioned adjacent the strings. This position is generally held by the user for an extended period of time, in many cases several hours. Individuals who play musical instruments and or carry out other activities were the muscles of the arm are required to support the arm in a constant a more or less constant position, can experience shoulder muscle strain or pain due to overuse of the particular muscles.

### SUMMARY OF THE INVENTION

The shoulder brace of the present invention is designed to allow a user to distribute a problematic force to a larger area of the shoulder and back (or to different larger muscles) to alleviate or significantly reduce possible muscle strain associated with the shoulder.

A shoulder brace according to the present invention provides assistance in reducing repetitive strain of arm and shoulder muscles of a user. The brace comprises a body portion for extending over the shoulders of a user and covering part of the upper back and chest of the user with at least one outer cuff secured to the body portion and having a lower edge adapted to engage and encircle the upper arm of the user at a position below the bleep.

The outer cuff includes a central load transferring strap extending across the shoulder joint and down an outside surface of the upper arm to the lower edge of the outer cuff, a forward load transferring strap extending from the shoulder across the bicep to the lower edge of the outer cuff at an inside surface of the elbow, and a rear load transferring strap extending from the shoulder across a back surface of the arm to the lower edge of the outer cuff.

The body portion includes a shoulder portion extending from front to back across the top of the shoulder at a position inwardly of the shoulder joint. The shoulder portion is adapted to releasably connect with the load transfer straps via three adjustment tabs of the outer cuff. The adjustment tabs allow a user to connect the outer cuff to the shoulder portion in a desired support position of the outer cuff. With this arrangement a lift force for holding the upper arm can be distributed to the torso to assist arm and shoulder muscles that otherwise may be exposed or prone to repetitive strain.

According to an aspect of the invention, the shoulder brace includes an inner shoulder cuff portion forming a continuation of the body portion to collectively cover the shoulder and bicep of a user with the outer shoulder cuff exterior to the inner cuff portion and joined to the inner cuff portion at the lower edge.

In a further aspect of the invention, the shoulder brace is designed to be worn by a user and includes two inner cuff portions and two outer cuff portions and provides the capability of supporting both shoulders of a user.

In yet a further aspect of the invention, the brace includes an adjustable securing strap at the lower edge of the inner cuff portion.

In an aspect of the invention, the outer shoulder cuff includes three adjustable securing points at a top edge with one point aligned with the top of the shoulder, one point at a diagonal across a rear of the shoulder and one point at a diagonal across a front of the shoulder. Preferably, each of the securing points includes a reinforcing strip aligned with the attachment point and extending to the lower edge for adjusting the lower edge with movement of the attachment point on the body portion.

### BRIEF DESCRIPTION OF THE DRAWINGS

Preferred embodiments of the invention are shown in the drawings wherein:

FIG. 1 is a perspective view of a user wearing the shoulder brace with his arms positioned for playing a guitar;

FIG. 2 is a partial front perspective view showing the shoulder brace as worn by the user;

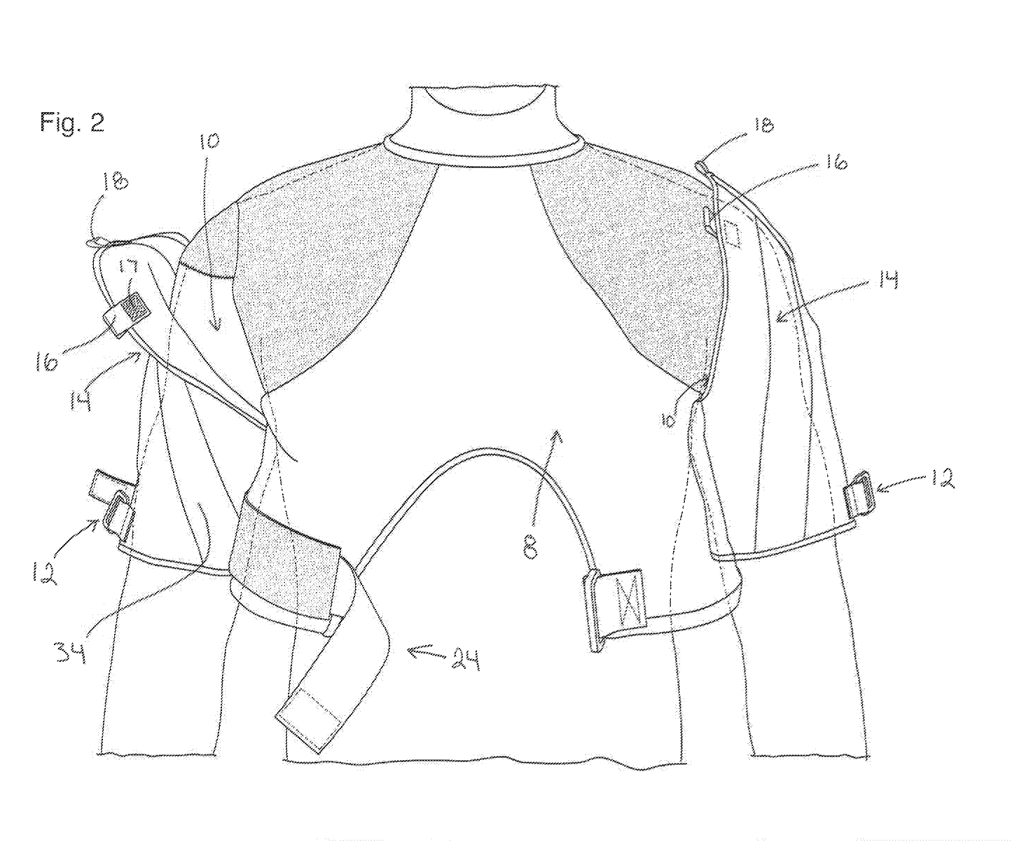

FIG. 3 is a perspective view similar to FIG. 2 with the shoulder brace being attached to the user's torso and the outer shoulder cuff of the shoulder brace attached in a particular position;

FIG. 4 is a partial perspective view showing the ability to adjust the shoulder cuff to redistribute a particular load to larger muscles adjacent the shoulder; and

FIG. 5 is a side perspective view of the brace as worn by a user.

### DETAILED DESCRIPTION OF THE PREFERRED EMBODIMENTS

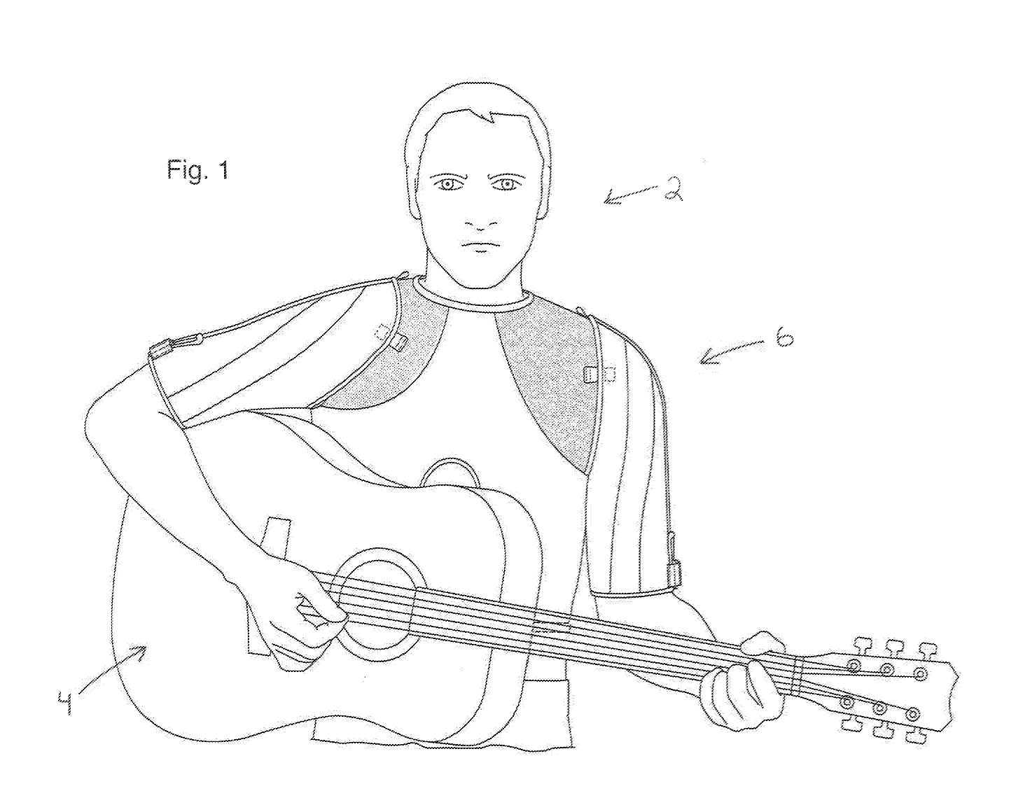

The user 2 as shown in FIG. 1, has the guitar 4 positioned for playing and is wearing the shoulder brace 6. The right arm of the user is in a somewhat raised position with the humorous portion of the user's arm resting on an upper surface of the guitar. The guitar is typically supported by a support strap that passes over one shoulder and down the back of the user.

Initially it is not difficult to maintain the user's arm position for playing of the guitar, however over extended periods of time the position of the right arm of the user as shown, can cause shoulder problems. For the user to play the guitar, he holds his arms in the positions generally shown in FIG. 1 and this requires a significant lifting of his right shoulder. The shoulder brace 6 is designed to allow the user to position and adjust, the shoulder brace to partially offset the force required to maintain this raised position of the shoulder and the arm and redistribute this force to a different portion of the shoulder or to the back of the user. The shoulder brace 6 as illustrated, supports both shoulders which may not be required for all applications. A brace capable of supporting both left and right hand users is more expensive to manufacture but avoids the necessity for two products or offering a single product to a smaller market size. The two sided brace also allows a user to provide a desired support for his opposite arm which is helpful for many applications.

The present brace allows a user to adjust an outer cuff of the shoulder brace to support a user's arm in a desired manner and to distribute this load or force to an area of the shoulder and/or back having larger muscles that are less prone to strain or damage. Each outer cuff is adapted to provide a number of different adjustments allowing a user to customize the amount support that he requires and to redistribute the load across the shoulder. This customised desired support is achieved while still allowing the user an effective range of motion and without affecting normal use of the equipment. It also allows further or ongoing adjustment when certain muscles become tired from prolonged use.

The shoulder brace 6 includes a body portion 8 having an adjustable body portion strap 24 to snug the body portion 8 about the torso of the user. Preferably the body portion is of a pull-on design. The body portion 8 also includes the shoulder portions 30 that are adapted to cooperate with the outer adjustable cuffs 14 that are attached to the inner arm cuffs 10 just below the bicep of the user. Adjustable arm cuff straps 12 allow the user to snug the inner arm cuffs (and the lower edge of the outer arm cuffs) about the arm at a position below the bicep. A lifting force is applied upwardly by the user during attachment of the outer arm cuff to the shoulder portions in a desired support position. The strap 12 secures both the inner and outer and cuffs below the bicep of the user anchoring the bottom edge of the cuffs.

The upper portion of the outer adjustable cuff 14 can be adjusted relative to the shoulder portions 30 and the outer adjustable cuff includes adjustable securing points 16, 18 and 20 for attaching the outer adjustable cuff 14 to the respective shoulder portion 30. Each of the adjustable points 16, 18 and 20 can easily be repositioned by the user using his other arm. Each of the adjustable points 16, 18 and 20 includes a Velcro portion 17 for securement to the shoulder portion 30.

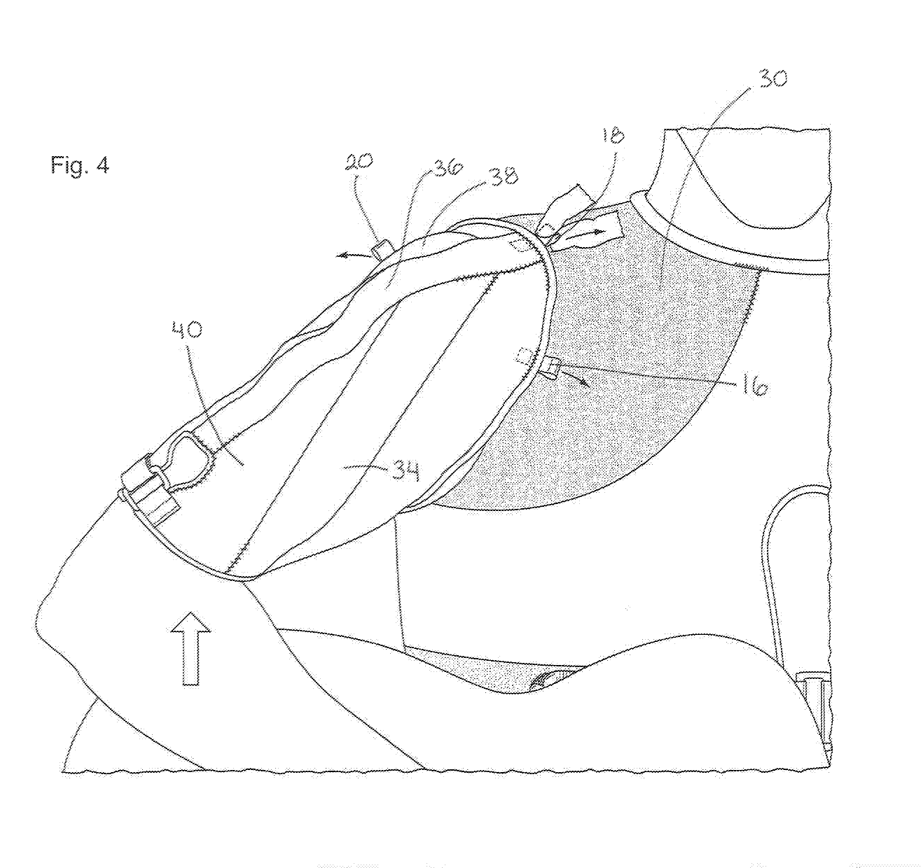

In this way the user is able to position the outer adjustable cuffs 14 on the shoulder portions 30 and apply a lifting force to the arm below the bleep as generally shown in FIG. 4. The user can continue to adjust the outer adjustable cuffs 14 until a desired support position is achieved and the brace serves to transfer or partially transfer the weight of the user's arm to a larger area of the shoulder or to the torso of the user. The muscles that are normally used to support this load are under reduced stress and are not prone to overexertion.

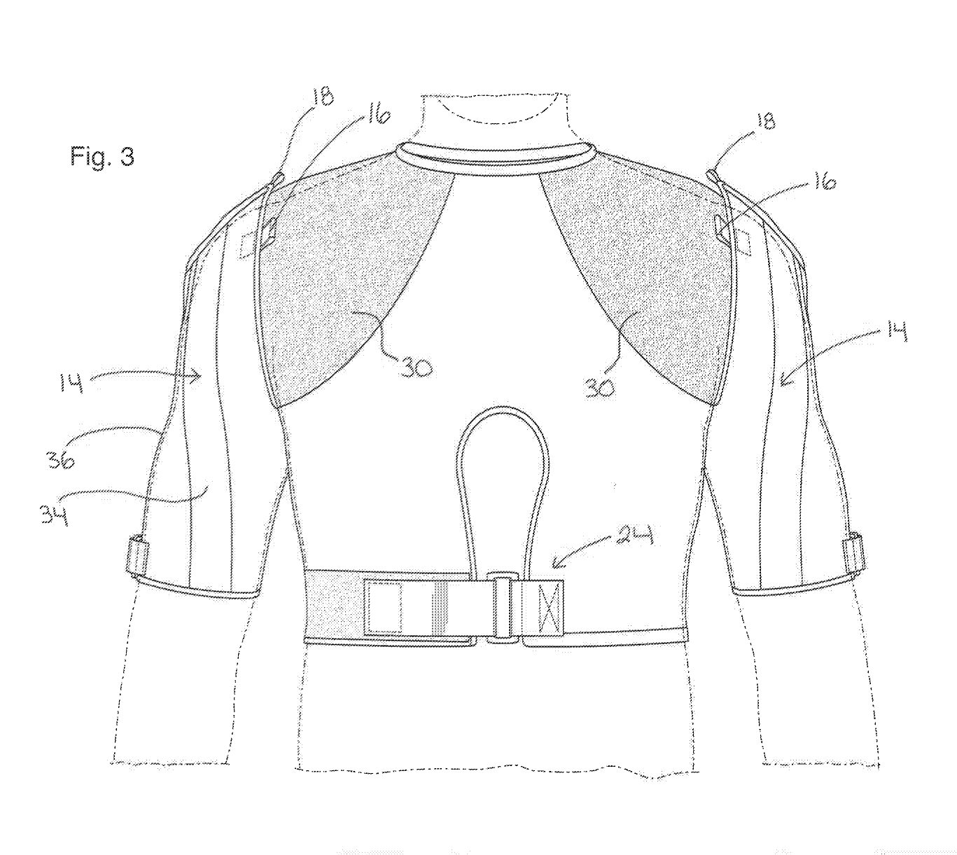

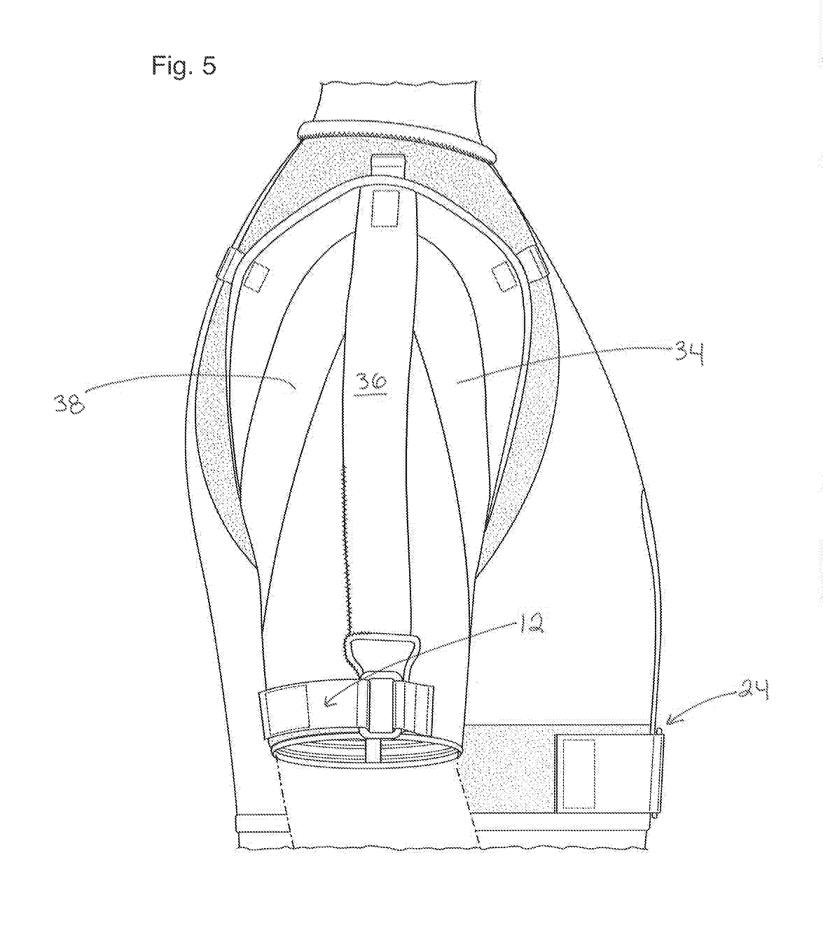

As shown in FIGS. 3, 4 and 5, the outer adjustable cuffs 14 include reinforcing segments 34, 36 and 38 which have some elasticity but are much less elastic than the body portion 40 (typically of a light weight elastic material for comfort) and the sleeve of the outer adjustable cuffs. The outer adjustable cuffs 14 include the reinforcing segments 34, 36 and 38 as shown in FIG. 5. Reinforcing segment 36 extends across the shoulder and down the outer portion of the arm and generally terminates at the adjustable arm cuff strap 12.

Adjustable point securement 18 is in line with this reinforcing segment and allows the user to effectively pull up on the outer portion of the arm by using the adjustment point securement 18 and attaching it to the shoulder portion. Reinforcing segments 34 and 38 extend from the upper portion of the shoulder downwardly with reinforcing segment 34 extending forwardly to cooperate with the arm cuff strap 12 and reinforcing segment 36 extending rearwardly to cooperate with the arm cuff strap as shown in FIG. 5.

With this arrangement the user can customize the support by using the adjustable point securements 16, 18 and 20 to apply arm lifting forces not only upwardly but forwardly and rearwardly. These forces are transmitted to the arm by the arm cuff strap 12 and through the reinforcing segments 34, 36 and 38 of the outer cuff to the shoulder and body portion of the brace. The lifting forces are transferred to large muscles of the user on his torso adjacent the shoulder. In this way, the user can customize the amount of lift that is being carried by the shoulder of the user without over exerting the muscles that would normally provide these forces. The lifting force is preferably redistributed to larger muscles of the user's chest and back. With the brace, the user is able to maintain a desired playing position without incurring repetitive muscle strain in his arm or shoulder.

Individual users may adjust the brace to assist with any pre-existing conditions they may have, as well as providing support for the particular session. The major muscles involved in using the brace are deltoid/supraspinatus (shoulder abductor-raises shoulder-rotator cuff)/biceps plus some shoulder capsule involvement.

The fabric of the body portion 8 is relatively elastic and lightweight to be comfortable for the user over an extended period of time. The purpose of the body portion 8 is to maintain the shoulder portions 30 in the desired position. This is achieved as the adjustable body portion strap 24 is in engaged about the torso of the user. The shoulder portions are of a higher strength material that also allows repetitive securement with the Velcro type fasters provided on the adjustable point securements 16, 18 and 20.

The shoulder brace has been used for individuals suffering from arm or shoulder pain associated with a known load supported for an extended period of time. The brace allows both a desired amount of preloading or support useful for repetitive guitar playing while also allowing the user to continue to perform in the normal manner. The shoulder brace does not appreciably restrict the movement of the user and is designed to effectively distribute a known force to the torso of the user. To a significant extent, the arm muscles of the user are bypassed to avoid arm or shoulder strain.

Although the brace has been designed for use in association with musical instruments, the brace can also be effectively used for other applications where a particular posture or position is to be maintained over a long period of time. For example, the shoulder brace may have application for individuals keyboarding for extended periods of time. Although the device is referred to as the shoulder brace, the device allows a user to customize the redistribution of a lift force that otherwise would be maintained by upper arm and shoulder muscles. The device distributes this lifting force to the user's back and front torso to reduce shoulder and upper arm strain.

Although preferred embodiments of the invention nave been described herein in detail, it is understood by those skilled in the art that variations may be made thereto without departing from the invention as defined by the claims.

The embodiments of the invention in which an exclusive property or privilege is claimed are defined as follows:

1. A shoulder brace for assisting in reducing repetitive strain of arm and shoulder muscles comprising a body portion for extending over the shoulders of a user and covering part of the upper back and chest of the user;
at least one outer cuff secured to said body portion and having a lower edge adapted to engage and encircle the upper arm of the user at a position below the bicep;
said outer cuff including a central load transferring strap extending across the shoulder joint and down an outside surface of the upper arm to said lower edge of said outer cuff, a forward load transferring strap extending from the shoulder across the bicep to the lower edge of said outer cuff at an inside surface of the elbow, and a rear load transferring strap extending from the shoulder across a back surface of the arm to said lower edge of said outer cuff;
said body portion including a shoulder portion extending from front to back across the top of the shoulder inwardly of the shoulder joint, said shoulder portion being adapted to releasably connect with said load transfer straps via three adjustment tabs of said outer cuff, said adjustment tabs allowing a user to connect said outer cuff to said shoulder portion in a desired support position of said outer cuff, whereby a lift force for holding the upper arm can be distributed to the torso to assist arm and shoulder muscles that otherwise may be exposed to repetitive strain.

2. A shoulder brace as claimed in claim 1 wherein said shoulder brace includes an inner shoulder cuff portion forming a continuation of said body portion to collectively cover the shoulder and bicep of a user with said outer shoulder cuff exterior to said inner cuff portion and joined to said inner cuff portion at said lower edge.

3. A shoulder brace as claimed in claim 2 wherein said shoulder brace is designed to be worn by a user and includes two inner cuff portions and two outer cuff portions and provides the capability of supporting both shoulders of a user.

4. A shoulder brace as claimed in claim 1 including an adjustable securing strap at said lower edge.

5. A shoulder brace as claimed in claim 4 wherein said outer shoulder cuff includes three adjustable securing points at a top edge with one point aligned with the top of the shoulder, one point at a diagonal across a rear of the shoulder and one point at a diagonal across a front of the shoulder.

6. A shoulder brace as claimed in claim 5 wherein each of said securing points includes a reinforcing strip aligned with the attachment point and extending to said lower edge for adjusting said lower edge with movement of said attachment point on said body portion.
